(19) United States
(12) Patent Application Publication
(10) Pub. No.: US 2011/0009617 A1
(43) Pub. Date: Jan. 13, 2011

(54) Ruthenium Catalysts and Uses Thereof

(76) Inventors: Chi-Ming Che, Hong Kong SAR (CN); Kwok-Wai Matthew Choi, Hong Kong (CN)

Correspondence Address: COOPER & DUNHAM, LLP, 30 Rockefeller Plaza, 20th Floor, NEW YORK, NY, 10112, US

(21) Appl. No.: 12/835,038

(22) Filed: Jul. 13, 2010

Related U.S. Application Data
(60) Provisional application No. 61/224,990, filed on Jul. 13, 2009.

(51) Int. Cl. C07D 205/00; B01J 31/06; C07D 265/30; C07D 498/04; C07D 207/00; C07D 305/12; C07C 69/76; B01J 31/28

(52) U.S. Cl. 540/200; 502/159; 544/171; 548/217; 548/556; 549/326; 560/103; 977/773; 977/775

(57) ABSTRACT

Ruthenium nanoparticles supported on non-cross-linked soluble polystyrene were prepared by reacting [RuCl₂(C₆H₅CO₂Et)]₂ with polystyrene in open air. They effectively catalyze intra- and intermolecular carbenoid insertion into C—H and N—H bonds, alkene cyclopropanation, and ammonium ylide/[2,3]-sigmatropic rearrangement reactions. This supported ruthenium catalyst is much more reactive than [RuCl₂(p-cymene)]₂ and Ru(Por)CO] for catalytic intermolecular carbenoid C—H bond insertion into saturated alkanes. By using a-diazoacetamide as a substrate for intramolecular carbenoid C—H insertion, the supported ruthenium catalyst can be to recovered and reused for ten successive iterations without significant loss of activity.

### CROSS REFERENCE TO RELATED APPLICATION

This application claims the benefit of U.S. Provisional Patent Application No. 61/224,990 filed on Jul. 13, 2009, the entire contents of which are incorporated by reference herein.

### BACKGROUND OF THE INVENTION

Transition-metal-mediated carbenoid transfer and insertion reactions are useful for the construction of carbon-carbon and carbon-heteroatom bonds.^{[1]}Rhodium(II),^{[2]} copper(I),^{[3]} and ruthenium(II)^{[4]} complexes have been proven to be effective catalysts for the decomposition of diazo compounds to generate reactive metallocarbene intermediates, which are directly responsible for these catalytic X—H bond formation reactions (X═C, Si, N, P, O, halides). ^{[1-4]} Previously we found that [RuCl₂(p-cymene)]₂ can effectively catalyze intermolecular carbenoid C—H insertion of a-diazoacetamides to cis-β-lactams with yields up to 97%.^{[5]} We also reported the synthesis of poly(ethylene glycol) (PEG)-supported ruthenium porphyrin complexes, which are suitable catalysts for epoxidation, cyclopropanation, and aziridination of alkenes.^{[6]} However, these PEG-supported ruthenium porphyrin complexes are inactive toward intra- and intermolecular carbenoid C—H insertion reactions.

In the context of developing catalytic carbenoid transfer reactions with practical applications, immobilization of metal catalysts on a solid support is a commonly employed strategy. The immobilization of rhodium (II),^{[7-9]} copper (I),^{[10]} and ruthenium (II)^{[11]} complexes on solid supports for heterogeneous catalytic carbenoid transfer reactions have been reported. In contrast, reports of carbenoid transfer to C═C bonds and insertion into C—H bonds employing metal catalysts supported on soluble polymer, which are bona fide homogeneous catalysts, are sparse.^{[12]} In this area, we are interested in non-cross-linked polystyrene (NCPS), which is commercially available, has the advantage of homogeneous solution chemistry (high reactivity and ease of analysis), and at the same time allows easy isolation and purification of the organic products.^{[13]}

Immobilization of a metal catalyst on polystyrene by microencapsulation was previously reported by Kobayashi and Akiyama.^{[14]} This immobilization strategy does not require ligand derivatization, allowing a convenient synthesis of polymer-supported metal catalysts. Herein we report that NCPS is an excellent carrier of ruthenium nanoparticles and that the resulting polymer-supported ruthenium nanoparticles are effective homogeneous catalysts for carbenoid transfer reactions with high substrate conversion and product turnover. This supported ruthenium catalyst shows good solubility in tetrahydrofuran, dichloromethane, chloroform, benzene, ethyl acetate, and toluene, but is insoluble in

hexane and methanol. It can be recovered, and its reuse has been demonstrated in catalytic intramolecular carbenoid C—H insertion reactions for ten iterations without loss of activity.

### SUMMARY OF THE INVENTION

The invention provides a method for making a soluble non-cross-linked polymer supporting ruthenium nanoparticle catalyst comprising reacting a soluble non-cross-linked soluble polymer supported ruthenium nanoparticles with RuCl₂:(C₆H₅CO₂Et) to form the soluble polymer supported ruthenium nanoparticle supported catalyst. The polymer may be polystyrene, poly(tert-butylstyrene) (NCPtBS), poly(tert-butylstyrene-co-styrene) (NCPtBS-co-PS), or poly(N-isopropylacrylamide).

The invention further provides a method for inserting carbenoids into α-diazo compounds comprising reacting an α-diazo precursor in the presence of a non-cross-linked soluble polystyrene supported nanoparticle catalyst.

### BRIEF DESCRIPTION OF THE DRAWINGS

Further features and advantages of the invention will become apparent upon review of the following detailed description of the preferred embodiments taken in connection with the attached drawings in which:

FIG. 1 illustrates the synthesis of polymer supported ruthenium catalyst NCPS-Ru.

FIG. 2 illustrates intramolecular tandem ammonium ylide formation/[2,3]-sigmatropic rearrangement reactions catalyzed by NCPS-Ru.

FIG. 3a is TEM images of ruthenium nanoparticles.

FIG. 3b is a histogram showing the distribution of the diameter of ruthenium nanoparticles.

FIG. 3c is a SAED pattern of the ruthenium nanoparticles.

FIG. 3d is a high resolution TEM image of a single ruthenium nanoparticle showing clear lattice fringes. The insert at top right corner is the FFT image of the particle. The insert at bottom right corner is the TEM image obtained by filtering the image corresponding to that nanoparticle with Gatan Digital Micrograph program.

FIG. 4 shows XPS spectra of (a) NCPS-Ru 1 and (b) the sample prepared by reacting NCPS and [RuCl₂(C₆H₅CO₂Et)]₂ in the presence of NaBH₄ in 1,2-dichloroethane.

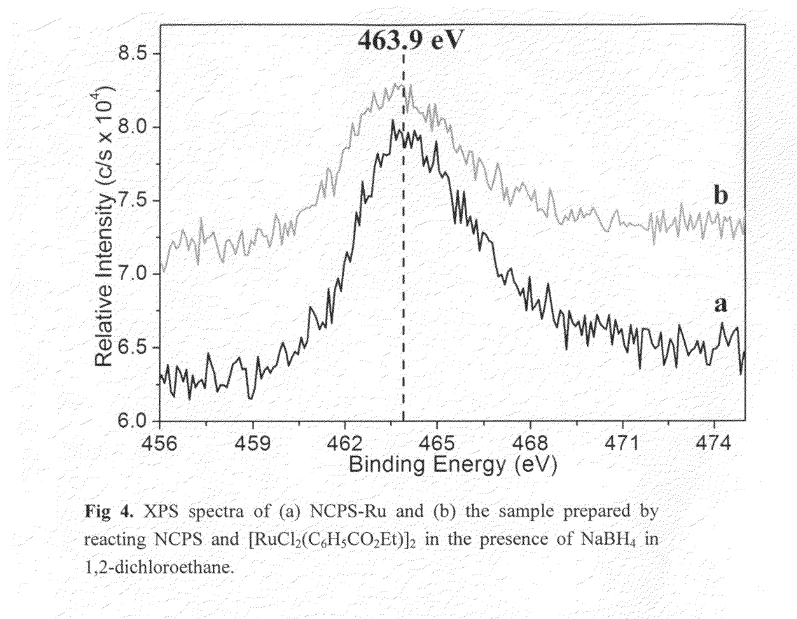

### DETAILED DESCRIPTION OF THE INVENTION

The present invention relates to the use of non-cross-linked soluble polystyrene supported ruthenium nanoparticles for practical carbenoid transfer reactions. In a typical example, preparation of the ruthenium nanoparticles catalyst NCPS-Ru was undertaken by heating a mixture of non-cross-linked polystyrene NCPS (0.50 g) and [RuCl₂(C₆H₅CO₂Et)]₂ (0.10 g, 0.16 mmol) in 1,2-dichloroethane (30 mL) in open air at 70° C. for 24 h (Scheme 1). The solution was concentrated in vacuo. The residue was taken up in 5 mL of 1,2-dichloroethane. This solution was added dropwise to a vigorously stirred cold hexane (200 mL). The black precipitate was filtered and dried to afford NCPS-Ru as a black powder (0.58 g, ca 100%). The ruthenium content of NCPS-Ru was determined to be 5.0% w/w using inductively coupled plasma spectroscopy (ICP), and hence the loading was 0.50 mmol/g.

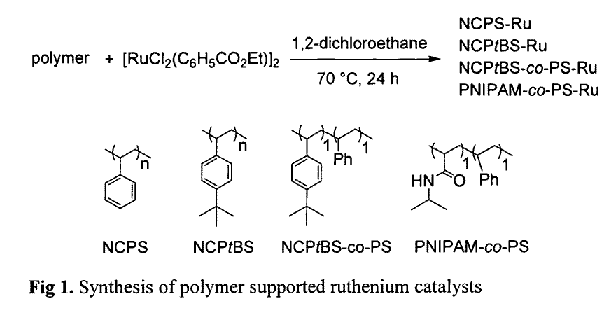

Similarly, other soluble polymer supports such as poly(tert-butylstyrene) (NCPtBS), poly(tert-butylstyrene-co-styrene) (NCPtBS-co-PS), and poly(N-isopropylacrylamide-co-styrene) (PNIPAM-co-PS) were used to react with [RuCl₂(C₆H₅CO₂Et)]₂ in 1,2-dichloroehtane to give the corresponding soluble polymer-supported ruthenium catalysts as depicted in FIG. 1.

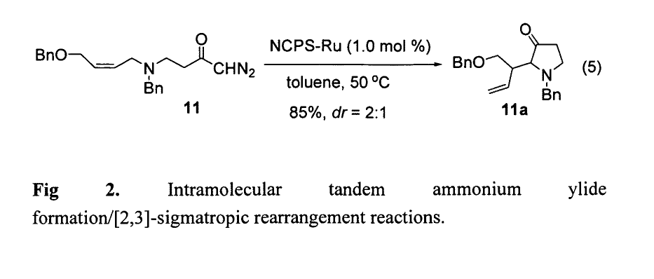

Examination of NCPS-Ru by transmission electron microscopy (TEM) showed the presence of well-dispersed spherical ruthenium nanoparticles (FIG. 2a) with the average diameter and monodispersity being 1.72±0.17 nm and 9.9%, respectively (FIG. 2b). Selected area electron diffraction (SAED) image showed two broad diffraction rings, which can be indexed back to metallic ruthenium (JCPDS no. 06-0663) (FIG. 2c). High resolution TEM imaging showed clear lattice fringes revealing that the nanoparticles were single-crystallized (FIG. 2d). The d-spacing of the particle was 2.047 Å corresponding to the (101) plane of metallic ruthenium.

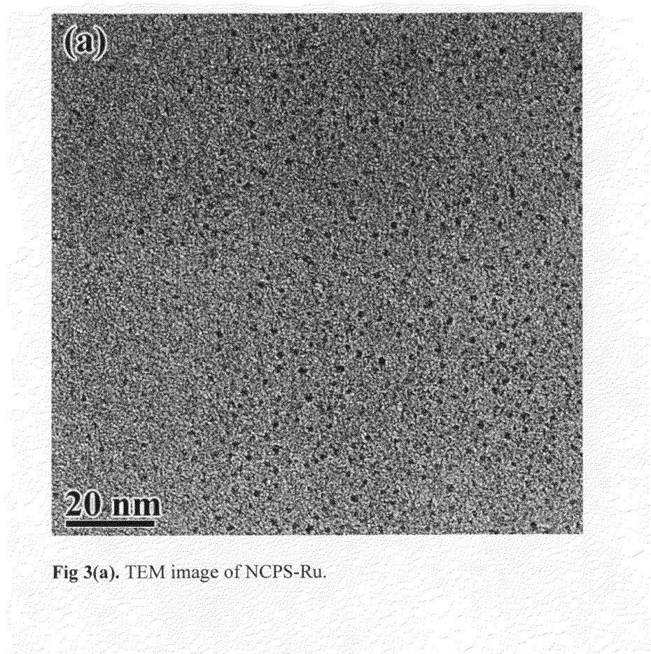

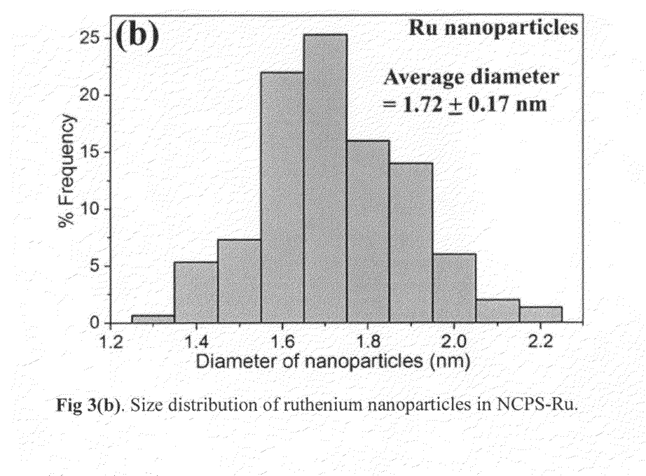

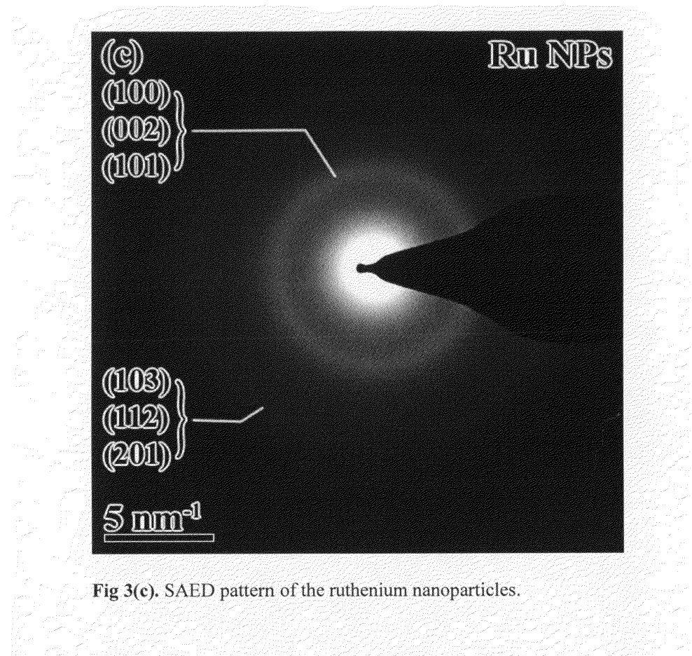

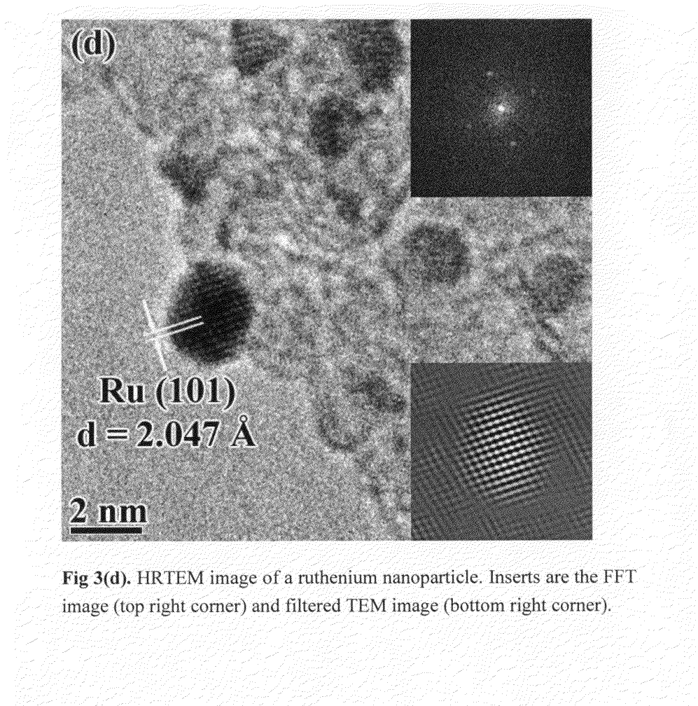

X-Ray photoelectron spectroscopic (XPS) analysis of a freshly prepared NCPS-Ru catalyst showed a peak at 463.9 eV corresponding to Ru 3p_{3/2} binding energy (see FIG. 3). This value slightly deviated from the reported value of bulk ruthenium metal (462.0 eV) [J. F. Moulder, W. F. Stickle, P. E. Sobol, K. D. Bomben is in Handbook of X-ray Photoelectron Spectroscopy (Eds.: J. Chastain, R. C. King), Physical Electronic, Eden Prairie, 1995], revealing that the polymer supported ruthenium nanoparticles might contain surface coated oxidized ruthenium ions.

### Example 1

### Intramolecular Carbenoid C—H Insertion of α-Diazoacetamides Catalyzed by NCPS-Ru

The invention further relates to synthesis of cis-β-lactams via intramolecular carbenoid C—H insertion of α-diazoacetamides catalyzed by NCPS-Ru. A mixture of diazo compound (1.0 mmol) and NCPS-Ru (1.0 mol %) was stirred in toluene (2 mL) at 70° C. The reaction was monitored by TLC analysis (20% EtOAc-hexane) for complete consumption of diazo starting material. Upon addition of hexane (2 mL) the reaction mixture was centrifuged, and aliquots were taken from the supernatant liquid for product identification and quantification by ¹H NMR spectroscopy. To obtain a pure product, the supernatant was separated and evaporated to dryness by rotary evaporation, and the residue was loaded onto a silica gel for column chromatography.

The substrate scope of the intramolecular carbenoid C—H insertions has been examined and the results are depicted in Table 1. A variety of substrates undergo cyclization in the presence of NCPS-Ru (1 mol %). The stereoselectivity is similar to that of homogeneous [RuCl₂(p-cymene)]₂ catalyst (M. K.-W. Choi, W.-Y. Yu, C.-M. Che, Org. Lett. 2005, 7, 1081). For example, effective transformation of α-diazoacetamides having different aryl substituents to the corresponding cis-β-lactamshave been achieved in >90% yields (entries 2 and 3). When α-diazoketone was employed as substrate, the NCPS-Ru-catalyzed intramolecular carbenoid C—H is insertion produced trans-β-lactam exclusively in 93% yield (entry 4). The carbenoid C—H insertion of N,N-diisopropyl substituted α-diazoacetamide was directed to the methine) (3° C.) c—H bond furnishing β-lactam in 89% isolated yield (entry 5). Interestingly, with the α-diazoanilide containing an electron donating group (OMe) or electron withdrawing group (NO₂), only intramolecular carbenoid C—H insertion to the phenyl group was found and the corresponding γ-lactams were isolated in good to excellent yields (entries 6 and 7). The catalysis could be performed in a preparative-scale (4.0 g substrate). With 1 mol % Ru catalyst, the diazo compounds were completely consumed within 4 h and cis-β-lactam (3.3 g) was formed as a 98% yield in a one-pot reaction.

### Example 2

### Recyclability of NCPS-Ru

The NCPS-Ru recovered from the intramolecular carbenoid C—H insertion reactions was mixed with diazo compound (1.0 mmol) in toluene (2 mL) at 70° C. The reaction was monitored by TLC analysis (20% EtOAc-hexane) for complete consumption of diazo starting material. Upon addition of hexane (2 mL), the reaction mixture was centrifuged, and aliquots were taken from the supernatant liquid for product identification and quantitation by ¹H NMR spectroscopy. The reaction vessel containing the catalyst was recharged with diazo compound and toluene (2 mL) for another consecutive reaction run.

The soluble polystyrene supported NCPS-Ru can be recovered and reused. The results in the recycling of NCPS-Ru for intramolecular carbenoid C—H insertions is are listed in Table 2. The NCPS-Ru was subjected to ten successive reuses under identical reaction conditions. The organic product was simply recovered by removing solvent from the filtrate without any further purification. After ten consecutive reactions, the recovered NCPS-Ru was found to contain 5.0 w/w of ruthenium based on ICP analysis. This ruthenium content was essentially the same as the initial value, revealing no detectable catalyst leaching over the 10 consecutive reactions.

### Example 3

### NCPS-Ru Catalyzed Intramolecular Carbenoid C—H Insertion of α-Diazo Compounds Derived from Various α-Amino Acids

The invention relates to synthesis of highly functionalized γ-lactams via intramolecular carbenoid C—H insertion of α-diazoacetamides catalyzed by NCPS-Ru. A mixture of diazo compound (1.0 mmol) and NCPS-Ru (1.0 mol %) was stirred in toluene (2 mL) at 70° C. The reaction was monitored by TLC analysis (20% EtOAc-hexane) for complete consumption of diazo starting material. Upon addition of hexane (2 mL), the reaction mixture was centrifuged, and aliquots were taken from the supernatant liquid for product identification and quantitation by ¹H NMR spectroscopy. To obtain pure product, the supernatant was separated and evaporated to dryness by rotary evaporation, and the residue was loaded onto silica gel for column chromatography.

Highly functionalized γ-lactams can be synthesized through catalytic carbenoid C—H insertion of diazoacetamides derived from amino acids (C. H. Yoon, D. L. Flanigan, B.-D. Chong, K. W. Jung, J. Org. Chem. 2002, 67, 6582). The results using NCPS-Ru as catalyst are depicted in Table 3. Treatment of diazoacetamide 5a prepared from L-phenylalanine with NCPS-Ru as catalyst (1 mol %) gave trans, trans-γ-lactam 6a in 89% yield. (Table 3, entry 1). The trans, trans-stereochemistry was established by ¹H-1H NOESY NMR analysis. Similarly, trans, trans-γ-lactam 6b was obtained in 90% yield using 5b as substrate (Table 3, entry 2). α-Diazoacetamide 5c containing an electron donating OTBS ether group also underwent Ru-catalyzed cyclization to afford a 1:1 mixture of diastereomeric bicyclic lactams in 92% yield (Table 3, entry 3). When the reaction was performed at lower temperature (40° C.), a mixture of diastereomers was obtained in an overall yield of 89% and with a ratio of 7c:6c=2:1 (Table 3, entry 4).

### Example 4

### NCPS-Ru Catalyzed Intermolecular C—H insertion of Hydrocarbons

The invention also relates to intermolecular C—H insertion of hydrocarbon with methyl phenyldiazoacetate catalyzed by NCPS-Ru. A mixture of methyl phenyldiazoacetate (1.0 mmol), 1,4-cyclohexadiene (2.0 mmol) and NCPS-Ru (1.0 mol %) was stirred in toluene (2 mL) at 70° C. for 2 h. Upon addition of hexane (2 mL), the reaction mixture was centrifuged and aliquots were taken from the supernatant liquid for product identification and quantitation by ¹H NMR spectroscopy. To obtain pure sample of the product, the supernatant liquid was separated and evaporated to dryness by rotary evaporation and the residue was loaded onto a silica gel column chromatography.

A mixture of methyl phenyldiazoacetate (1.0 mmol), 1,4-cyclohexadiene (2.0 mmol) and MCPS-Ru (1.0 mol %) was stirred in neat hydrocarbon (2 mL) at 70° C. for 12 h. Upon addition of hexane (2 mL), the reaction mixture was centrifuged and aliquots were taken from the supernatant liquid for product identification and quantitation by ¹H NMR spectroscopy. To obtain pure sample of the product, the supernatant liquid was separated and evaporated to dryness by rotary evaporation and the residue was loaded onto a silica gel column chromatography.

Due to its inherent difficulty, intermolecular carbenoid C—H insertion of saturated alkanes is more challenging to accomplish [C. Jia, T. Kitamura, Y. Fujiwara, Acc. Chem. Res. 2001, 34, 633; V. Ritleng, C. Sirlin, M. Pfeffer, Chem. Rev. 2002, 102, 1731], and there has been no example on the use of ruthenium complexes as catalysts for intermolecular carbenoid insertion to saturated alkanes. In this work, we investigated NCPS-Ru catalyzed intermolecular carbenoid C—H insertion reactions, and the results are depicted in Table 4. The reaction of methyl phenyldiazoacetate with neat cyclohexane in the presence of NCPS-Ru (1.0 mol %) afforded the C—H insertion product in 60% yield. Similarly, the NCPS-Ru catalyzed reaction of methylphenyldiazoacetate with 1,4-cyclohexadiene furnished the C—H insertion product and cyclopropanated product with a ratio of 4:1 in 66% overall yield (Table 4, entry 2). With ethyl benzene, a 1.2:1 mixture of insertion products were obtained in 63% overall yield (Table 4, entry 3).^{[15]} The reaction of indane with methyl to phenyldiazoacetate gave the C—H insertion product 17 as a single isomer in 62% yield (Table 4, entry 4). With n-hexane as substrate, the secondary (18a and 18b) to primary (19) C—H insertion products were formed with a ratio of 5:1 in 50% overall yield (Table 4, entry 5).

### Example 5

### NCPS-Ru Catalyzed Intramolecular Cyclopropanation of Allyl-Diazoacetates

The invention relates to intramolecular cyclopropanation of allyl diazoacetates catalyzed by NCPS-Ru. To a solution of NCPS-Ru (1.0 mol %) in toluene (2 mL) was added dropwise a solution of allyl diazoacetate (1.0 mmol) in toluene (2 mL), over 10 h at 70° C. After the addition, stirring was continued until all the diazo compounds had been consumed. Upon addition of hexane (2 mL) the reaction mixture was centrifuged, and aliquots were taken from the supernatant liquid for product identification and quantitation by ¹H NMR spectroscopy. To obtain pure product, the supernatant was separated and evaporated to dryness by rotary evaporation, and the residue was loaded onto a silica gel for column chromatography.

The NCPS-Ru catalyst is also active toward intramolecular cyclopropanation of alkenes (Table 5). Treatment of a variety of allyl diazoacetates with the catalyst led to the corresponding cyclopropyl lactones in good yields after 12 h (70%-89%, see Table 5). The catalyst could be recovered quantitatively by precipitation and filtration.

### Example 6

### NCPS-Ru Catalyzed Intramolecular Tandem Ammonium Ylide Formation/[2,3]-Sigmatropic Rearrangement Reactions

The invention relates to intramolecular tandem ammonium ylide formation/[2,3]-sigmatropic rearrangement reactions catalyzed by NCPS-Ru. To a solution of NCPS-Ru (1.0 mol %) in toluene (2 mL) was added dropwise a solution of diazo compound (1.0 mmol) in toluene (2 mL) over 2 h at 50° C. After the addition, stirring was continued until all the diazo compounds had been consumed. Upon addition of hexane (2 mL) the reaction mixture was centrifuged, and aliquots were taken from the supernatant liquid for product identification and quantitation by ¹H NMR spectroscopy. To obtain pure product, the supernatant was separated and evaporated to dryness by rotary evaporation, and the residue was loaded onto a silica gel for column chromatography.

We have also examined intramolecular tandem ammonium ylide formation/[2,3]-sigmatropic rearrangement reactions. Treatment of 8 with NCPS-Ru 1 (1.0 mol %) afforded [2,3]-sigmatropic rearrangement product 8a in 92% yield without any [1,2]-rearrangement product being detected (Table 6, entry 1). This result is comparable to the finding using [Ru^{II}(TTP)(CO)] as catalyst (C.-Y. Zhou, W.-Y. Yu, P. W. H. Chan, C.-M. Che, J. Org. Chem. 2004, 69, 7072). Similarly diazoketone 9 was found to undergo effective cyclization to give pyridone 9a in 89% yield (Table 6, entry 2) and diazoester 10 was converted to morpholinone 10a in 91% yield (Table 6, entry 3). Previously, we reported that the [Ru^{II}(TTP)(CO)] to catalyzed intramolecular ammonium ylide/[2,3]-sigmatropic rearrangement could be applied as a key step for the total synthesis of (±)-platynecine. In this work we found that using NCPS-Ru 1 catalyst (1 mol %), a comparable result (85% yield, dr=2:1) was obtained (Scheme 2).

### Example 7

### NCPS-Ru Catalyzed Intermolecular Cyclopropanation of Alkenes

The invention relates to intermolecular cyclopropanation of alkene with ethyl diazoacetate catalyzed by NCPS-Ru. To a solution of NCPS-Ru (1.0 mol %) and alkene (2 mmol) in toluene (2 mL) was added dropwise a solution of allyl diazoacetate (1.0 mmol) in toluene (2 mL) over 10 h at 70° C. After the addition, stirring was continued until all the diazo compounds had been consumed. Upon addition of hexane (2 mL) the reaction mixture was centrifuged, and aliquots were taken from the supernatant liquid for product identification and quantitation by ¹H NMR spectroscopy. To obtain a pure product, the supernatant was separated and evaporated to dryness by rotary evaporation, and the residue was loaded onto a silica gel for column chromatography.

In turning our attention to intermolecular carbenoid transfer reactions, we examined intermolecular cyclopropanation, N—H insertion and C—H insertion using NCPS-Ru as catalyst. The supported catalyst NCPS-Ru is active toward intermolecular cyclopropanation of alkenes, as revealed by the results depicted in Table 7.

### Example 8

### NCPS-Ru Catalyzed Intermolecular N—H Insertion of Amines

The invention further relates to intermolecular N—H insertion of amine with ethyl diazoacetate catalyzed by NCPS-Ru. Ethyl diazoacetate (1.0 mmol) was added in one portion to a mixture of amine (1.1 mmol) and NCPS-Ru (1.0 mol %) in toluene (2 mL) at 70° C. After the addition, stirring was continued until all of the diazo compounds had been consumed. Upon addition of hexane (2 mL) the reaction mixture was centrifuged, and aliquots were taken from the supernatant liquid for product identification and quantitation by ¹H NMR spectroscopy. To obtain a pure product, the supernatant was separated and evaporated to dryness by rotary evaporation, and the residue was loaded onto a silica gel for column chromatography.

The carbenoid insertion into N—H bonds is an attractive carbenoid transformation for the synthesis of α-amino carboxylic compounds.^{[16]} In this work, we found that the NCPS-Ru catalyzed intermolecular N—H insertion reactions could be performed without using a slow addition procedure or an inert atmosphere. The N—H insertion products were obtained in high yields by one pot reaction of amine and ethyl diazoacetate in toluene at 70° C. in open atmosphere (i.e. without Ar/N₂ protection). Complete substrate conversion was observed within 1 h (Table 8). No diazo compound coupling product was formed.

In the literature, most reported metal-catalyzed intermolecular N—H insertion reactions using diazo compounds were conducted in a millimole scale.^{[16]} In this work, we have examined the feasibility of scaling up the intermolecular N—H insertion reaction of aniline and ethyl diazoacetate using 0.1 mole of substrate. (Table 8, entry 8). Ethyl diazoacetate (0.10 mol) was added in one portion to a mixture of aniline (0.11 mol) and NCPS-Ru (0.1 mol %) in toluene at 70° C. in open atmosphere. Complete substrate conversion was formed within 1 h. N-phenylglycine ethyl ester was obtained in 97% yield. At a lower catalyst loading (0.01 mol %) intermolecular N—H insertion in a 0.1 mole scale took a longer reaction time (4 days) for complete consumption of ethyl diazoacetate, no diazo coupling products (fumarte/maleate) were detected by ¹H NMR analysis of the reaction mixture. N-phenylglycine ethyl ester was obtained in 93% yield.

All of the references cited herein are incorporated by reference:
[1] a) M. P. Doyle, M. A. McKervey, T. Ye, Modern Catalytic Methods for Organic Synthesis with Diazo Compounds, Wiley, New York, 1998;
b) M. P. Doyle, D.C. Forbes, Chem. Rev. 1998, 98, 911-936;
c) H. M. L. Davies, R. E. J. Beckwith, Chem, Rev. 2003, 103, 2861-2904;
d) V. F. Ferreira, Curr. Org. Chem. 2007, 11, 177-193;
e) H. M. L. Davies, S. J. Hedley, Chem. Soc. Rev. 2007, 36, 1109-1119.
a) P. M. P. Gois, C. A. M. Afonso, Eur. J. Org. Chem. 2004, 3773-3788; b) H. M. L. Davies, Angew. Chem. 2006, 118, 6574-6577; Angew. Chem. Int. Ed. 2006, 45, 6422-6425;
c) A. G. H. Wee, Curr. Org. Synth. 2006, 3, 499-555; d) H. M. L. Davies, J. R. Manning, Nature 2008, 451, 417-424.
[3] a) M. M. DPaz-Requejo, P. J. PQrez, J. Organomet. Chem. 2005, 690, 5441-5450; b) M. M. DPaz-Requejo, T. R. Belderrain, M. C. Nicasio, P. J. PQrez, Dalton Trans. 2006, 5559-5566.
[4] a) F. Kakiuchi, N. Chatani in Ruthenium in Organic Synthesis (Ed.:S.-I. Murahashi), Wiley-VCH, Weinheim, 2004, pp. 219-256;
b) G. Maas, Chem. Soc. Rev. 2004, 33, 183-190;
c) C.-M. Che, C.-M. Ho, J.-S. Huang, Coord. Chem. Rev. 2007, 251, 2145-2166.
[5] M. K.-W. Choi, W.-Y. Yu, C.-M. Che, Org. Lett. 2005, 7, 1081-1084.
[6] J.-L. Zhang, C.-M. Che, Org. Lett. 2002, 4, 1911-1914.
[7] a) M. P. Doyle, D. J. Timmons, J. S. Tumonis, H.-M. Gau, E. C. Blossey, Organometallics 2002, 21, 1747-1749; b) M. P. Doyle, M. Yan, H.-M. Gau, E. C. Blossey, Org. Lett. 2003, 5, 561-563.
[8] a) T. Nagashima, H. M. L. Davies, Org. Lett. 2002, 4, 1989-1992; b) H. M. L. Davies, A. M. Walji, Org. Lett. 2003, 5, 479-482; c) H. M. L. Davies, A. M. Walji, T. Nagashima, J. Am. Chem., Soc. 2004, 126, 4271-4280; d) H. M. L. Davies, A. M. Walji, Org. Lett. 2005, 7, 2941-2944.
[9] a) J. Lloret, F. Estevan, K. Bieger, C. Villanueva, M. A. Rbeda, Organometallics 2007, 26, 4145-4151; b) J. Lloret, M. Stern, F. Estevan, M. Sanaffl, M. A. Rbeda, Organometallics 2008, 27, 850-856.
[10] a) J. M. Fraile, J. I. GarcPa, J. A. Mayoral, M. RoldTn, Org. Lett. 2007, 9, 731-733; b) J. M. Fraile, J. I. GarcPa, J. A. Mayoral, Coord. Chem. Rev. 2008, 252, 624-646.
[11] a) N. E. Leadbeater, K. A. Scott, L. J. Scott, J. Org. Chem. 2000, 65, 3231-3232; b) A. Cornejo, J. M. Fraile, J. I. GarcPa, M. J. Gil, S. V. Luis, V. MartPnez-Merino, J. A. Mayoral, J. Org. Chem. 2005, 70, 5536-5544; c) A. Cornejo, J. M. Fraile, J. I. GarcPa, M. J. Gil, V.
MartPnez-Merino, J. A. Mayoral, Tetrahedron Lett. 2005, 61, 12107-12110; d) G. Simonneaux, P. L. Maux, Y. Fonand J. Rault-Berthelot, Coord. Chem. Rev. 2006, 250, 2212-2221.
[12] a) M. P. Doyle, M. Y. Eismont, D. E. Bergbreiter, H. N. Gray, J. Org. Chem. 1992, 57, 6103-6105; b) D. E. Bergbreiter, J. Tian, Tetrahedron Lett. 2007, 48, 4499-4503; c) M. Glos, O. Reiser, Org. Lett. 2000, 2, 2045-2048.
[13] For reviews on soluble polymers supports, see: a) D. E. Bergbreiter, Chem. Rev. to 2002, 102, 3345-3384; b) T. J. Dickerson, N. N. Reed, K. D. Janda, Chem. Rev. 2002, 102, 3325-3344.
[14] S. Kobayashi, R. Akiyama, Chem. Commun. 2003, 449-460.
[15] H. M. L. Davies, Q. Jin, P. Ren, A. Y. Kovalevsky, J. Org. Chem. 2002, 67, 4165-4169.
[16] B. Liu, S.-F. Zhu, W. Zhang, C. Chen, Q.-L. Zhou, J. Am. Chem. Soc. 2007, 129, 5834-5835.

TABLE 1
NCPS—Ru-catalyzed intramolecular carbenoid C—H insertion of
α-diazoacetamides.^{[a]}
(2)

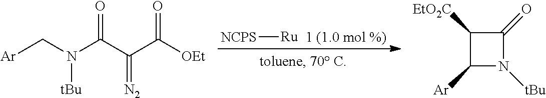

| Entry | Substrate | Product | Yield [%]^{[b]} |
|---|---|---|---|
| 1 | 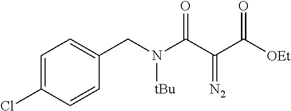 | 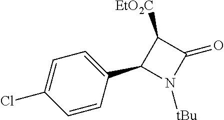 | 98 |
| 2 | 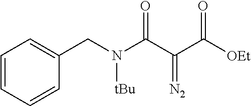 | 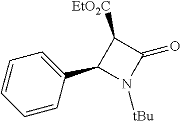 | 90 |
| 3 | 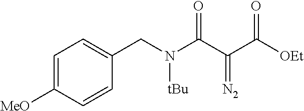 | 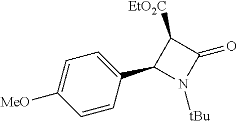 | 91 |
| 4 | 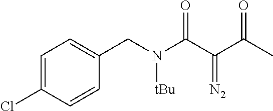 | 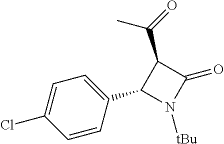 | 93 |
| 5 | 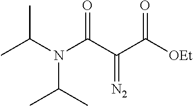 | 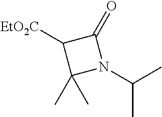 | 89 |
| 6 | 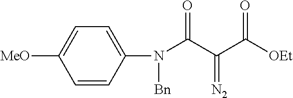 | 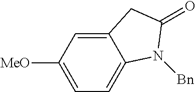 | 93 |
| 7 | 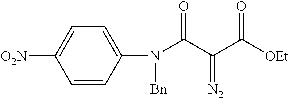 | 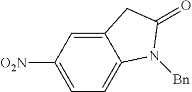 | 78 |

^{[a]}A mixture of diazo compound (1.0 mmol) and NCPS—Ru 1 (1.0 mol %) was stirred in toluene at 70° C. in an open atmosphere.
^{[b]}Isolated yield.

TABLE 2
Recyclability of NCPS—Ru toward intramolecular carbenoid C—H
insertion reaction.^{[a]}

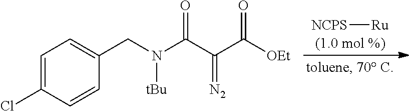

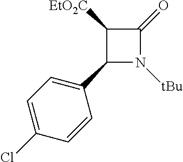

| | Time | Conv | Yield |
|---|---|---|---|
| Cycle | [h] | [%] | [%]^{[b]} |
| 1 | 0.5 | 100 | 97 |
| 2 | 0.5 | 100 | 94 |
| 3 | 0.5 | 100 | 99 |
| 4 | 0.5 | 100 | 98 |
| 5 | 0.5 | 100 | 96 |
| 6 | 0.5 | 100 | 94 |
| 7 | 0.5 | 100 | 93 |
| 8 | 0.5 | 100 | 95 |
| 9 | 0.5 | 100 | 97 |
| 10 | 0.5 | 100 | 94 |

^{[a]}A mixture of diazo compound (1.0 mmol) and NCPS—Ru 1 (1.0 mol %) was stirred in toluene at 70° C. in an open atmosphere.
^{[b]}Yield of products was determined by ¹H NMR using 1,1-diphenylethene as internal standard.

TABLE 3
NCPS—Ru catalyzed intramolecular carbenoid C—H insertion of
α-diazo-compounds derived from various α-amino acids.^{[a]}
(3)

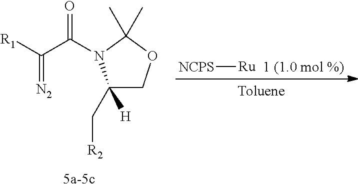

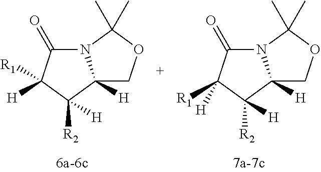

| | | | Diazo | Temp | Time | | Yield |
|---|---|---|---|---|---|---|---|
| Entry | R₁ | R₂ | (5) | [° C.] | [h] | 6:7^{[c]} | [%] |
| 1 | EtO₂C | Ph | 5a | 70 | 48 | 6a | 89 |
| 2 | MeCO | Ph | 5b | 70 | 48 | 6b | 90 |
| 3 | MeCO | OTBS | 5c | 70 | 16 | 1:1 | 92 |
| 4 | MeCO | OTBS | 5c | 40 | 48 | 1:2 | 89 |

^{[a]}A mixture of diazo compound (1.0 mmol) and NCPS—Ru 1 (1.0 mol %) was stirred in toluene at 70° C. under nitrogen atmosphere.
^{[b]}Isolated yields.
^{[c]}The stereoselectiviry of 6 and 7 were characterized by 2D-NOESY NMR study.

TABLE 4
Intermolecular C—H insertion of hydrocarbon with methyl
phenyldiazoacetate catalyzed by NCPS—Ru.^{[a]}

| Entry | Substrate | Product | Yield [%]^{[b]} |
|---|---|---|---|
| 1 | 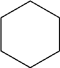 | 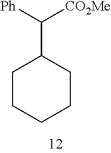 | 60 |
| 2^{[c]} | 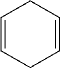 | 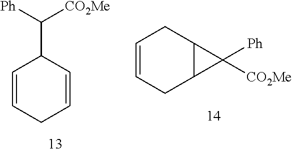 | 66 (4:1) |
| 3 | 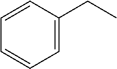 | 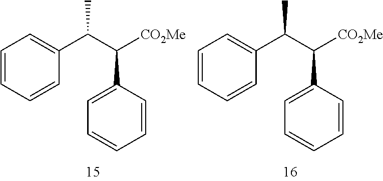 | 63 (1.2:1) |
| 4 | 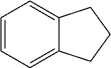 | 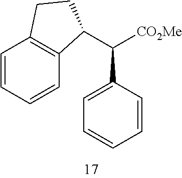 | 62 |
| 5 | 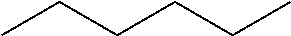 | 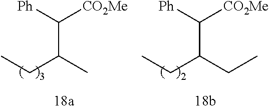 | 50^{[d]} |
| | | 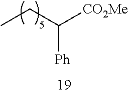 | |

^{[a]}A mixture of diazo compound (1.0 mmol) and NCPS—Ru 1 (1.0 mol %) was stirred in neat substrate at 70° C. under nitrogen atmosphere.
^{[b]}Isolated yield.
^{[c]}Toluene as solvent.
^{[d]}(18a + 18b):19 = 5:1

TABLE 5
NCPS—Ru catalyzed intramolecular cyclopropanation of
allyl-diazoacetates.^{[a]}
(4)

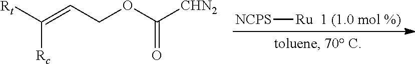

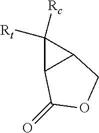

| Entry | Rₜ | R_{c} | Yield [%]^{[b]} |
|---|---|---|---|
| 1 | H | H | 86 |
| 2 | CH₃ | H | 82 |
| 3 | CH₃CH₂ | H | 83 |
| 4 | Ph | H | 89 |
| 5 | H | Ph | 70^{[c]} |
| 6 | H | CH₃CH₂ | 70 |
| 7 | CH₃ | CH₃ | 85 |

^{[a]}A allyl diazoacetates (1.0 mmol) was dropwise addition to NCPS—Ru 1 (1.0 mol %) in toluene at 70° C. under nitrogen atmosphere.
^{[b]}Isolated yield.
^{[c]}syn:anti = 4:1.

TABLE 6
NCPS—Ru catalyzed intramolecular tandem ammonium ylide
formation/[2,3]-sigmatropic rearrangement reactions.^{[a]}

| Entry | Substrate | Product | Yield [%]^{[b]} |
|---|---|---|---|
| 1 | 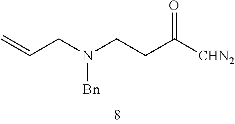 | 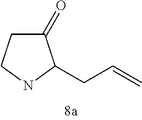 | 92 |
| 2 | 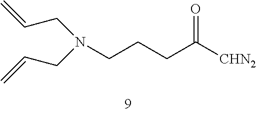 | 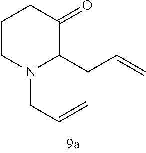 | 89 |
| 3 | 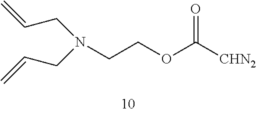 | 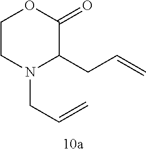 | 91 |

^{[a]}A diazo compound (1.0 mmol) was dropwise addition to NCPS—Ru 1 (1.0 mol %) in toluene at 50° C. under nitrogen atmosphere.
^{[b]}Isolated yield.

TABLE 7
NCPS—Ru 1 catalyzed Intermolecular cyclopropanation of alkene
with ethyl diazoacetate.^{[a]}

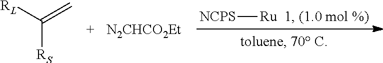

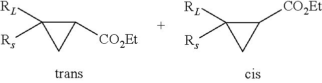

| Entry | R_{L} | Rₛ | Yield [%]^{[b]} | trans:cis^{[c]} |
|---|---|---|---|---|
| 1 | C₆H₅ | H | 91 | 70:30 |
| 2 | p-Cl—C₆H₄ | H | 85 | 73:27 |
| 3 | p-OMe—C₆H₄ | H | 90 | 68:32 |
| 4 | n-butyl | H | 67 | 70:30 |

^{[a]}A EDA (1.0 mmol) was dropwise addition to a mixture of alkene (2.0 mmol) and NCPS—Ru 1 (1.0 mol %) in toluene at 70° C. under nitrogen atmosphere.
^{[b]}Isolated yield.
^{[c]}trans:cis ratio was determined by ¹H NMR.

TABLE 8
NCPS—Ru 1 catalyzed intermolecular N—H insertion of amines^{[a]}

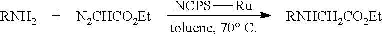

| Entry | Substrate | Product | Yield [%]^{[b]} |
|---|---|---|---|
| 1 | 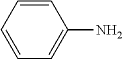 | 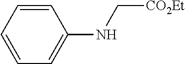 | 99 |
| 2 | 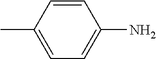 | 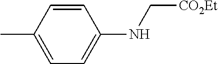 | 97 |
| 3 | 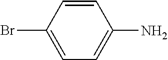 | 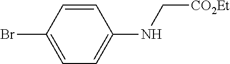 | 97 |
| 4 | 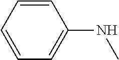 | 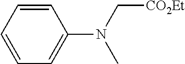 | 83 |
| 5 | 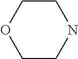 | 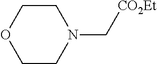 | 91 |
| 6 | 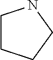 | 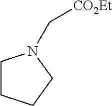 | 89 |
| 7 | 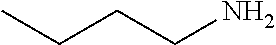 | 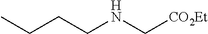 | 60 |
| 8 | 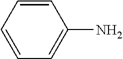 | 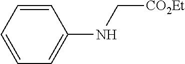 | 97 |

^{[a]}A EDA (1.0 mmol) was added in one portion to a mixture of amine (1.1 mmol) and NCPS—Ru 1 (1.0 mol %) in toluene at 70° C. in open atmosphere.
^{[b]}Isolated yield.
^{[c]}A EDA (0.10 mol) was added in one portion to a mixture of aniline (0.11 mol) and NCPS—Ru 1 (0.1 mol %) in toluene at 70° C. in open atmosphere.

We claim:

1. A method for making non-cross-linked soluble polymer supported ruthenium nanoparticles, comprising: heating a mixture of non-cross-linked polymer nanoparticles with RuCl₂(C₆H₅CO₂Et) to form non-cross-linked soluble polymer supported ruthenium nanoparticles.

2. The method of claim 1, wherein the non-cross linked polymer support is polystyrene, poly(tert-butylstyrene) (NCPtBS), poly(tert-butylstyrene-co-styrene) (NCP tBS-co-PS), or poly(N-isopropylacrylamide).

3. The method of claim 2, wherein the non-cross-linked soluble polymer is polystyrene.

4. The method of claim 3, wherein heating is carried out in the presence of dichloroethane and sodium borohydride.

5. A non-cross-limbed soluble polymer supported ruthenium nanoparticle catalyst made in accordance with the method of claim 3.

6. A method for inserting carbenoids into α-diazo compounds comprising reacting an α-diazo precursor in the presence of a non-cross linked soluble polystyrene supported ruthenium nanoparticle catalyst made in accordance with the method of claim 3.

7. A method for synthesizing γ-lactams comprising reacting a diazoacetamide precursor in the presence of a non-cross linked soluble polystyrene supported ruthenium nanoparticle catalyst.

8. A method for intermolecular C—H insertion of hydrocarbon into a saturated alkane comprising reacting a methyl phenyldiazene with a saturated alkane in the presence of a non-cross linked soluble polystyrene supported ruthenium nanoparticle catalyst.

9. A method for intramolecular cyclopropanation of an allyl-diazoacetate comprising reacting the allyl-diazoacetate in the presence of a non-cross linked soluble polystyrene supported ruthenium nanoparticle catalyst made in accordance with the method of claim 3.

10. A method for intramolecular cyclopropanation of an alkene comprising reacting the alkene in the presence of a non-cross linked soluble polystyrene supported ruthenium nanoparticle catalyst made in accordance with the method of claim 3.

11. A method for an intramolecular tandem ammonium ylide formation/[2-3]-sigmatropic rearrangement reaction comprising adding a diazo compound to a solution of a non-cross linked soluble polystyrene supported ruthenium nanoparticle catalyst made in accordance with the method of claim 3.

12. A method of intermolecular N—H insertion of amine comprising adding ethyl diazoacetate to a mixture of an amine and a non-cross linked soluble polystyrene supported ruthenium nanoparticle catalyst made in accordance with the method of claim 3.

13. A method in accordance with claim 12, wherein the reaction occurs in toluene.
